(19) United States
(12) Patent Application Publication
(10) Pub. No.: US 2019/0151242 A1
(43) Pub. Date: May 23, 2019

(54) LIPOSOME COMPOSITION FOR RELIEVING SYMPTOMS OF PARKINSON'S DISEASE AND ALZHEIMER'S DISEASE

(71) Applicants: Jeng-Fong CHANG, Taipei City (TW); Hui KUNG, Taipei City (TW)

(72) Inventor: Hui KUNG, Taipei City (TW)

(21) Appl. No.: 16/194,070

(22) Filed: Nov. 16, 2018

(30) Foreign Application Priority Data
Nov. 17, 2017 (TW) 106139972

(51) Int. Cl. A61K 9/127; A61K 31/197; A61K 31/202; A61K 47/10; A61K 31/685; A61K 47/26

(52) U.S. Cl. CPC A61K 9/127; A61K 31/197; A61K 47/26; A61K 47/10; A61K 31/685; A61K 31/202

(57) ABSTRACT

The present invention is to provide a liposome composition, comprising: a liposome enclosed by a phospholipid bilayer, wherein the phospholipid bilayer defines a receiving space therein; nutrients provided in the receiving space, wherein the nutrients comprising glutamic acid, docosahexaenoic acid (DHA), and lecithin; and a targeting substance embedded in or bound to the liposome. The liposome composition disclosed herein has a phospholipid bilayer and a special targeting substance that can pass through the BBB of a living body and then release nutrients, including glutamic acid, to activate glial cells in the living body, in order for the glial cells to acquire calcium ions in the living body, the goal being to promote synaptogenesis and to modulate neuronal plasticity and synaptic transmission while enhancing the glutamic acid-glutamine cycle.

### BACKGROUND OF THE INVENTION

### 1. Technical Field

The present invention relates to a liposome composition and more particularly to one for relieving the symptoms of Parkinson's disease and of Alzheimer's disease.

### 2. Description of Related Art

Parkinson's disease (PD) is a degenerative disease of the central nervous system resulting from deficiencies in the synthesis and action of dopamine and tends to impair a patient's mobility and verbal abilities. Alzheimer's disease (AD), on the other hand, is associated with cerebral degeneration and deteriorates slowly over time, causing persistent neural dysfunction, some late symptoms of which are incapability to speak normally, loss of long-term memory, difficulties in self-care, and abnormal behaviors. While many a research team has made great efforts studying PD, AD, and other central nervous system disorders, there is still no cure for those diseases. Most of the symptom relief treatments either have side effects or are not as effective as intended.

The unsatisfactory treatment results are attributable mainly to the blood-brain barrier (BBB), a self-protection mechanism of the human body. Lying between the brain and blood vessels therein, the BBB selectively blocks most of the drugs and proteins from entering the brain and is therefore an obstacle to be overcome by neurologists. In fact, one major objective of the development of neurological treatments is to find methods for breaking through the BBB in an effective, and more importantly safe and reversible, way. The BBB is composed of endothelial cells of the brain. These cells form layers of membranes that are wrapped tightly around each blood vessel in the brain so that bacteria, viruses, and other harmful substances in the blood are kept from entering the brain. The BBB, however, also blocks most of the drugs: only 25% or so of known medicines have access to the brain, which makes the treatment of brain-related diseases extremely difficult.

The conventional medicines for treating central nervous system diseases are delivered to the brain through the nasal cavity or other mucosa tissues; nevertheless, the multilayer tissues of the nasal cavity tend to stop macromolecular medicines from reaching the brain, which includes bones, the dura mater, and the arachnoid mater (a constituent part of the BBB). This is the fundamental reason why brain diseases are so hard to treat.

### BRIEF SUMMARY OF THE INVENTION

Take the treatment of PD for example. The conventional approaches are invasive and involve direct injection of the glial cell-derived neurotrophic factor (GDNF). One major side effect of such treatments is that trauma and complications are very likely to take place while the medicine is delivered to the brain. The conventional approaches, therefore, demand improvement.

In view of this, how to eliminate the aforementioned defects is the technical difficulty that the inventor of the present invention desires to solve. Therefore, the inventor of the present invention has been painstakingly researched for many years based on his years of experience in the art. After the improvement, the present invention was finally success and the present invention can enhance the effect.

One objective of the present invention is to provide a liposome composition, comprising: a liposome enclosed by a phospholipid bilayer, wherein the phospholipid bilayer defines a receiving space therein; nutrients provided in the receiving space, wherein the nutrients comprising glutamic acid, docosahexaenoic acid (DHA), and lecithin; and a targeting substance embedded in or bound to the liposome.

Further, the liposome is selected from the group consisting of: a liposome, a phytosome, an ethosome, a phosphatidylcholine, a phosphatidylserine, and a phosphatidylinositol.

Further, the liposome comprising a plurality of phospholipid bilayers that are enclosed to form a plurality of receiving spaces containing nutrients.

Further, the nutrients further comprises a vitamin, an antioxidant, or a mixture or a complex thereof

Further, the targeting substance is a vitamin E derivative, or a lipid derivative group, bound with a polyethylene glycol (PEG), or a derivative thereof, and glutathione (GSH), or a derivative thereof.

Further, the liposome is a targeting glutathione transporter.

Further, the liposome is a glutathione transporter targeted at the BBB.

Further, the PEG or PEG derivative comprises a carboxylic acid, maleimide, amide, or biotin.

Further, the targeting substance is polysorbate nanoparticles.

Further, the targeting substance is an amino acid sequence.

The liposome composition disclosed herein has a phospholipid bilayer and a special targeting substance that can pass through the BBB of a living body and then release nutrients, including glutamic acid, to activate glial cells in the living body, in order for the glial cells to acquire calcium ions in the living body, the goal being to promote synaptogenesis and to modulate neuronal plasticity and synaptic transmission while enhancing the glutamic acid-glutamine cycle.

### BRIEF DESCRIPTION OF THE SEVERAL VIEWS OF THE DRAWINGS

FIG. 1 shows a perspective, cross-sectional view of a preferred embodiment of the present invention.

FIG. 2 shows a cross-sectional view of a preferred embodiment of the present invention.

FIG. 3(a) to (c) show schematic views showing the effects of the preferred embodiment of the present invention.

FIG. 4 shows a DNA fragmentation image of a disease model mouse used in an embodiment of the present invention.

FIG. 5 shows the test result of Example 1 of the present invention.

FIG. 6 shows a survival analysis of Example 2 of the present invention.

### DETAILED DESCRIPTION OF THE INVENTION

The details and technical solution of the present invention are hereunder described with reference to accompanying drawings. For illustrative sake, the accompanying drawings are not drawn to scale. The accompanying drawings and the scale thereof are not restrictive of the present invention.

The use of “comprise” means not excluding the presence or addition of one or more other components, steps, operations, or elements to the described components, steps, operations, or elements, respectively. Similarly, “comprise,” “comprises,” “comprising” “include,” “includes,” and “including” are interchangeable and not intended to be limiting. As used herein and in the appended claims, the singular forms “a,” “an,” and “the” include plural referents unless the context dictates otherwise. The terms “a”, “an,” “the,” “one or more,” and “at least one,” for example, can be used interchangeably herein. The terms “about”, “approximately”, or “substantially” means having a value or range that is close to the allowable specified error to avoid any unreasonable third party illegally or unfairly using from understanding the precise or absolute value disclosed herein.

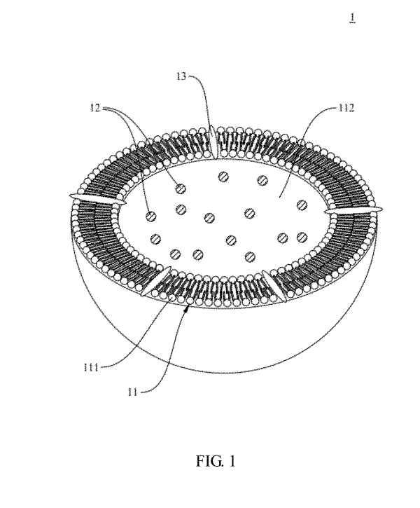

Referring to FIG. 1, the present invention provides a liposome composition 1, comprising: a liposome 11 enclosed by a phospholipid bilayer 111, wherein the phospholipid bilayer 111 defines a receiving space 112 therein; nutrients 12 provided in the receiving space 112, wherein the nutrients 12 comprising glutamic acid, docosahexaenoic acid (DHA), and lecithin; and a targeting substance 13 embedded in or bound to the liposome 11.

As used herein, the term “liposome 11” refers to a microspherical structure characterized mainly by a phospholipid bilayer 111. The phospholipid bilayer 111 forms the outermost layer of and thus encloses the microspherical structure, and inside the microspherical structure is a receiving space 112 surrounded by the phospholipid bilayer 111. The phospholipid bilayer 111 is composed of phospholipid molecules, wherein each phospholipid molecule has a hydrophobic end and a hydrophilic end. More specifically, the hydrophobic end of each phospholipid molecule is connected to that of another to form a phospholipid bi-molecule, with one of the hydrophilic ends facing outward. The phospholipid bilayer 111 is formed of a plurality of phospholipid bi-molecules arranged side by side in a spherical configuration and thus gives the liposome 11 its spherical structure. In one embodiment, the liposome 11 is selected from the group consisting of: a liposome, a phytosome, an ethosome, a phosphatidylcholine, a phosphatidylserine, and a phosphatidylinositol.

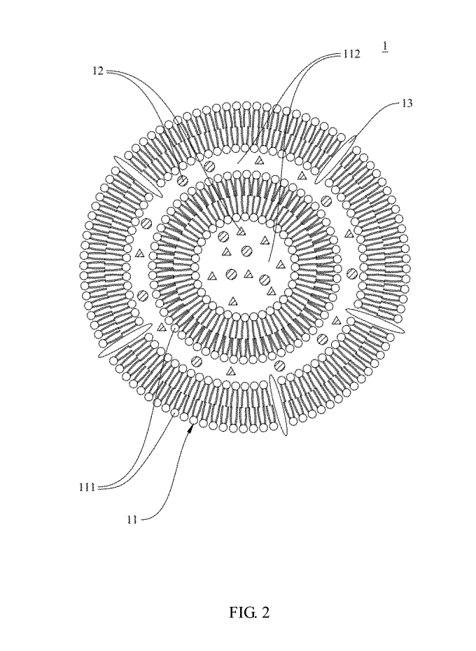

The liposome disclosed herein may be a small unilamellar vesicle (SUV), a large unilamellar vesicle (LUV), or a multilamellar vesicle (MLV). SUVs generally have a diameter of 15-30 nm and can be prepared by ultrasonication using a cup-horn, bath, or probe-tip ultrasonicator. LUVs have a diameter of 100-200 nm or more and can be prepared by extrusion, detergent-based dialysis, reverse evaporation, or ethanol injection. LUVs are stable during storage, whereas SUVs tend to fuse with one another spontaneously at a temperature lower than the lipid transition temperature required for forming liposomes. MLVs can be prepared from SUVs and LUVs, with amphiphilic lipids hydrated to form large “onion-like” structures. In one embodiment, the liposome of the present invention includes a plurality of phospholipid bilayers that are enclosed to form a plurality of receiving spaces, wherein the receiving spaced containing a certain amount of nutrients. FIG. 2, for example, shows an MLV with a plurality of nutrient-containing vesicular spaces.

As used herein, the term “nutrients 12” refers to substances that are provided in the receiving space 112 and are to be administered to a living body in order to be absorbed by the living body and achieve their intended effects. The nutrients 12 include glutamic acid, docosahexaenoic acid (DHA), and lecithin. In one embodiment, the nutrients 12 further include a vitamin, an antioxidant, or a mixture thereof. Besides, the liposome 11 in one embodiment includes only one phospholipid bilayer 111, and the nutrients 12 are contained in the receiving space 112 surrounded by the phospholipid bilayer 111. In another embodiment, by contrast, the liposome 11 includes a plurality of phospholipid bilayers 111, and the nutrients 12 are contained in the plural receiving spaces 112 surrounded by the phospholipid bilayers 111.

As used herein, the term “targeting substance 13” refers to a modifier on the liposome 11, wherein the modifier may be embedded in or bound to the liposome 11. In one embodiment, the targeting substance 13 includes an organic compound, a vitamin, a peptide, a hapten, an antibody, a phytohemato-agglutinin, a peptide hormone, an amino acid, a protein, a carbohydrate, or a derivative of any of the above. In a preferred embodiment, the targeting substance 13 is a vitamin E derivative (or a lipid derivative group) bound with a polyethylene glycol (PEG) (or a derivative thereof) and glutathione (GSH) (or a derivative thereof). In a more preferred embodiment, and by way of example only, the vitamin E derivative is a tocopherol, such as an α-tocopherol,5,7,8-trimethyltocol, a β-tocopherol,5, 8-dimethyltocol, a δ-tocopherol,8-methyltoco, a γ-tocopherol,7,8-dimethyltocol, a related salt, or the like. In a still more preferred embodiment, the PEG or PEG derivative includes a carboxylic acid, maleimide, amide, or biotin. In another embodiment, the targeting substance is polysorbate nanoparticles. In yet another embodiment, the targeting substance is an amino acid sequence.

Continued from the foregoing definition of the targeting substance 13, the liposome 11 in a preferred embodiment is a targeting glutathione transporter. In a more preferred embodiment, the liposome 11 is a glutathione transporter targeted at the BBB.

Generally speaking, the substances stated herein can pass through the BBB, either from blood to brain tissues or vice versa, by diffusion or mediated transport. Substances that can pass through the BBB by diffusion are mainly water and gases. Fat-soluble substances and fat-soluble solvents can easily pass through lipophilic cytomembranes and can therefore diffuse into the brain rapidly. Glucose, amino acids, and various ions, on the other hand, rely on mediated transport. A mediated transport system for glucose has stereospecificity, so only D-glucose is allowed to enter the brain; L-glucose cannot. As to amino acids, they enter brain tissues at different speeds, depending on whether a corresponding amino acid transporter is present and the quantity and quality (specificity) of the transporter. Most of the nutritionally essential amino acids can be transported rapidly, and all the amino acids that pass through the BBB with difficulty are nonessential ones. Ions are transported into brain tissues at different speeds, too, and generally much slower than into other tissues. It is worth mentioning that substances diffusible into the brain pass through the BBB at a lower speed once dissociated into ions. For example, NH₃, undissociated salicylic acid, and CO₂ enter the brain faster than NH₄⁺, salicylate ions, and HCO₃⁻, respectively. In addition, H⁺ is transported very slowly and provides a marked contrast to the rapidly diffusing CO₂. Noticing this helps understand the disparity between blood pH and brain tissue pH and why pCO₂ of blood is a better indicator of the acidity or alkalinity of brain tissues than blood pH.

Nanoparticles feature long life cycles, invisibility, and stereo-stability in the human body, and all these features help target substances in the human body. In a preferred embodiment, polysorbate nanoparticles are used to modify the surface of the disclosed liposome (i.e., serving as the targeting substance), in order for the liposome composition of the present invention to break through the BBB, have a higher concentration in the brain, and thereby enhance the effectiveness of treatment for patients with PD or AD. Oral administration of nanoscale lipids or polymeric nanoparticles allows these particles to adsorb better to the epithelial cells of the intestinal tract, be absorbed for a longer period of time, and thus enter the circulatory system more efficiently through the lymphatic system and through the ingestion of M cells of the Payer's patches in the small intestine.

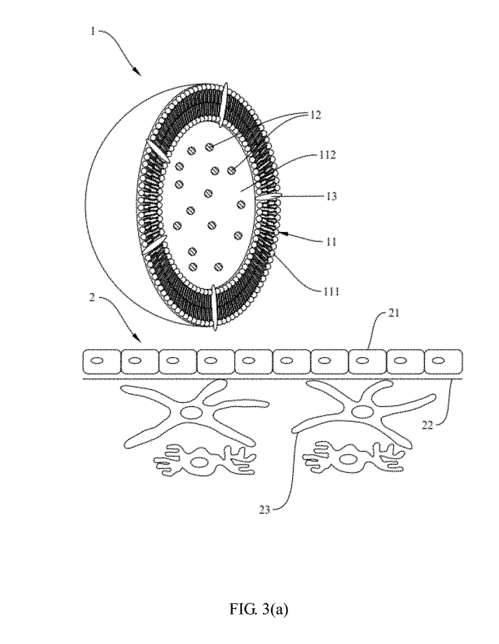

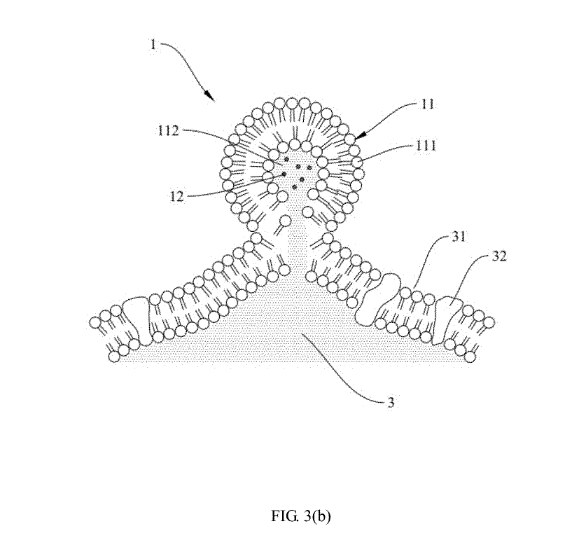

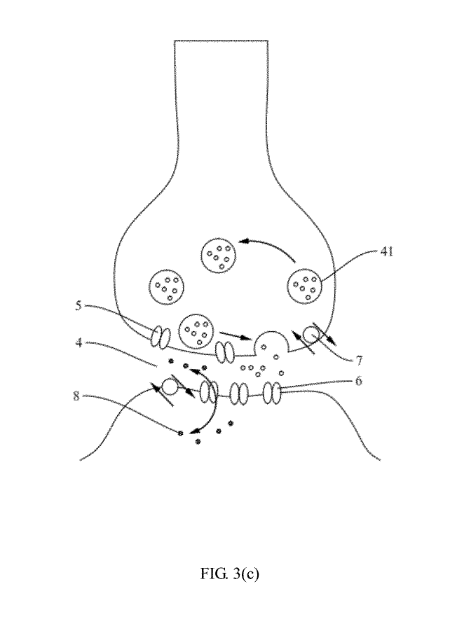

As used herein, the term “glutamic acid-glutamine cycle” refers to the following. Referring to FIG. 3(a) to FIG. 3(c), glutamic acid enters the BBB 2 through the endothelial cells 21 and the basement membrane 22 and is subsequently recycled by the glutamate transporters on the astrocytes 23 in the glial cells. The recycled glutamic acid is then converted by the astrocytes 23 into glutamine, with catalysis by glutamine synthetase. After that, the astrocytes 23 release the glutamine into the extracellular fluid, and the glutamine transporters on the neurons recycle the glutamine back into the nerve cells. Glutamine can be recycled by two types of neurons, namely the glutamic-acid neurons and the α-aminobutyric-acid neurons.

When recycled by the glutamine transporters on the glutamic-acid neurons, glutamine will be converted back into glutamic acid, with catalysis by glutaminase.

When recycled by the glutamine transporters on the α-aminobutyric-acid neurons instead, glutamine will first be converted into glutamic acid, with catalysis by glutaminase, and then form α-aminobutyric acid through synthesis catalyzed by glutamic acid decarboxylase. The resulting α-aminobutyric acid can be sent into synaptic vesicles 41 for storage, waiting to be released.

The intra-terminal concentration of glutamic acid is in direct proportion to the amount of α-aminobutyric acid stored in the synaptic vesicles 41. Glutamic acid recovered through the foregoing cycle can provide about 60% of the α-aminobutyric-acid neuron synapses 4 with the α-aminobutyric acid they are supposed to transmit. By achieving a balance between glutamic acid and α-aminobutyric acid, neurotransmission through the excitatory and inhibitory synapses 4 of the central nervous system can be stabilized to alleviate the symptoms of PD and AD.

As used herein, the term “PD symptom model” refers to a biological model capable of simulating the symptoms of PD. In the experiments performed for the present invention, as described further below, LRRK2^{R1441G} BAC transgenic mice were used because mutation of the LRRK2 gene is the most common hereditary factor of human PD. Researches have shown that BAC transgenic mice with an LRRK2 mutant (R1441G) can exhibit certain major characteristics of PD such as notable mobility impairment and pathological changes in the brain.

As used herein, the term “cylinder test” refers to a simple and highly effective test for assessing mobility impairment of PD mice, conducted according to the method described by Simon P. Brooks and Stephen B. Dunnett in “Tests to assess motor phenotype in mice: a user's guide”, Nature Neuroscience. More specifically, a mouse is placed in a transparent cylinder of a diameter of 12 cm and photographed from the front. The mobility of the mouse is assessed by counting horizontal limb movements of the mouse over 2 to 5 minutes, during which the mouse is fed vertically. The test is easy to carry out, can make accurate assessment, and does not require special training.

A detailed description and example embodiments of the present invention are given below. However, the example embodiments are for easier illustrating and are not limited to the scope of the present invention.

Disease Models: Transgenic Mice

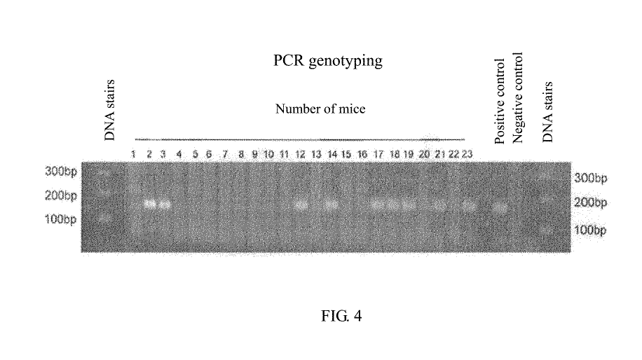

The LRRK2^{R1441G} BAC transgenic mice used in the experiments were purchased from the Jackson Laboratory and were raised in the laboratory animal room of the State University of New York in accordance with protocols approved by the Institutional Animal Care Use Committee (IACUC). Genotyping was performed on 10-day-old mice through biopsies of living tissues of the tails. The genome DNA of the tail tissues were extracted with the D Neasy Blood & Tissue Kit of QIAGEN, Germany. LRRK2^{R1441G} BAC was verified through 154-bp gene segment amplification using two gene-specific primers (TGA TTC TCG TTG GCA CAC AT and GCC AAA GCA TCA GAT TCC TC) and the polymerase chain reaction (PCR) cycle (94° C. for 45 seconds, 58° C. for 45 seconds, and 72° C. for 45 seconds; 35 repetitions) of the Mastercycler of Eppendorf, Germany. Referring to FIG. 4 for some DNA segment images of the transgenic mice, the 154-bp transgenic DNA segments of the 2^{nd}, 3^{rd}, 12^{th}, 14^{th}, 17^{th}, 18^{th}, 19^{th}, 21^{st}, and 23^{rd} LRRK2^{R1441G} transgenic mice were PCR amplified. The foregoing transgenic mice were used in the experiments described below when they were 8-12 months old, serving as experimental models for PD simulation.

Healthy Models: Wild Mice

In contrast to the disease models, wild non-transgenic mice 8-12 months of age were used as the healthy models, or the control group, in the following experiments.

Method of Administration: Oral Gavage

Oral gavage is a common and convenient way to administer drugs to mice in an experiment, carried out through a plastic tube that guides liquid medicine directly from the mouth of a mouse into its stomach. To prevent misplacement of the plastic tube and to keep the tube from puncturing the trachea, the experiments for the present invention used the flexible and bulb-tip plastic feeding tubes designed by Instech Solomon, USA. This administration method not only simulates how people take drugs orally, but also allows the dosage and administration time to be controlled with precision to facilitate study and comparison. In the experiments described below, drugs were administered to the mice once daily for two weeks in a row, and the drug administration procedure is as follows:

To begin with, an operator pinches the skin on the shoulders of a mouse tightly using the thumb and middle finger. Then, the operator pulls the head and neck of the mouse with the index finger to straighten the esophagus. After that, the bulb tip of the plastic tube is put into the mouth of the mouse, pushed gently down the esophagus along the right-hand side of the back of the pharynx, and then eased into the stomach, into which a 200-mL mixed liquor (as detailed below with reference to embodiment 1 and comparative examples 1 and 2) is subsequently injected through the tube.

### I. Embodiment 1: Liposome Composition

Referring to FIG. 1 and FIG. 3(a) to FIG. 3(c), the liposome composition 1 in embodiment 1 includes: a liposome 11 enclosed by a phospholipid bilayer 111, wherein the phospholipid bilayer 111 defines a receiving space 112 therein; nutrients 12 provided in the receiving space 112, wherein the nutrients 12 include glutamic acid, DHA, and lecithin; and a targeting substance 13 bound to the liposome 11, wherein the targeting substance 13 is composed of a vitamin E derivative (or a lipid derivative group) bound with a PEG and GSH.

The targeting substance 13 on the liposome composition 1 targets, and subsequently binds with, the receptor 32 (glutathione transporter) on the cell membrane 31 of the BBB 2 such that the phospholipid bilayer 111 of the liposome composition 1 passes through the BBB 2 and enters the cell 3 through endocytosis, releasing the nutrients 12 in the receiving space 112. The glutamic acid in the nutrients 12 goes through the in vivo glutamic acid-glutamine cycle and binds with the ion channel 5, thereby opening the ion channel 5 and allowing calcium ions 8 to flow in through the ion channel 5.

A phosphate-buffered saline (PBS) containing 10 mg of the liposome composition 1 was mixed with sunflower oil at a ratio of 1:1 to produce 200 mL of mixed liquor.

### II. Comparative Example 1: Excipient

For comparative example 1, a PBS free of the disclosed liposome composition was mixed with sunflower oil at a ratio of 1:1 to produce 200 mL of mixed liquor. Comparative example 1 was different from embodiment 1 in that the former lacked the liposome composition of the present invention.

### III. Comparative Example 2: Liposome Composition Containing Only Glutamic Acid as Nutrients

Comparative example 2 used the disclosed liposome composition, and yet the nutrients in the composition included only glutamic acid. A PBS containing 10 mg of such a liposome composition (containing only glutamic acid as the nutrients) was mixed with sunflower oil at a ratio of 1:1 to produce 200 mL of mixed liquor. Comparative example 2 was different from embodiment 1 in that the former lacked the nutrients DHA and lecithin.

[Experiment 1]: Mobility Assessment after Administration of Liposome Composition

Four first-generation mice that were born in the same litter and raised in the laboratory animal room till 8 months old were used in experiment 1, including a healthy-model wild mouse and three disease-model transgenic mice. The healthy-model wild mouse and one of the disease-model transgenic mice were administered with the mixed liquor of comparative example 1, whereas the other two disease-model transgenic mice were administered with the mixed liquor of embodiment 1. Each of the four mice was then subjected to a preliminary cylinder test for three times.

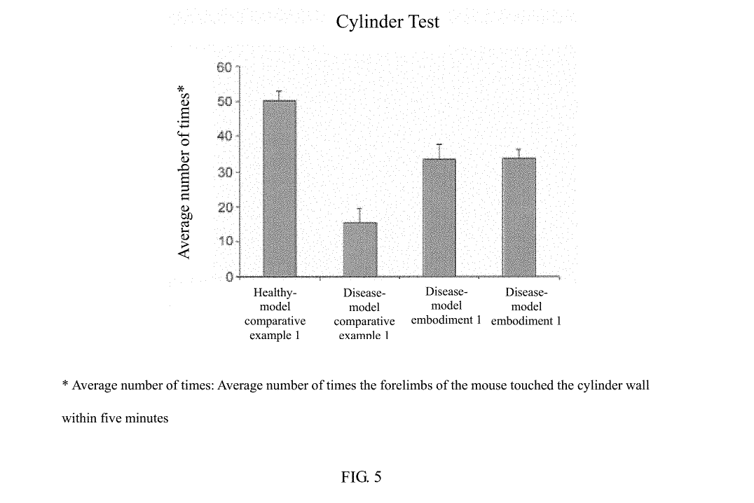

FIG. 5 shows the average numbers of times for which the forelimbs of the mice touched the cylinder wall within five minutes, along with their respective error bars. In the group of mice that were administered with the mixed liquor of comparative example 1, the disease-model mouse exhibited significantly lower mobility (P=0.018) than the same-litter healthy-model mouse. On the other hand, the disease-model mice that were administered with the mixed liquor of embodiment 1 exhibited significantly higher mobility (P=0.03, P=0.06) than the disease-model mouse that was administered with the mixed liquor of comparative example 1. That is to say, of the disease-model mice, those administered with the mixed liquor of embodiment 1 showed a significant relief of PD symptoms (i.e., an increase of mobility) as compared with that administered with the mixed liquor of comparative example 1.

[Experiment 2]: Mobility Assessment after Administration of Liposome Compositions Containing Different Nutrients

Two first-generation mice that were born in the same litter and raised in the laboratory animal room till 8 months old were used as the disease-model transgenic mice in experiment 2. One of the mice was administered with the mixed liquor of comparative example 2, and the other mouse was administered with the mixed liquor of embodiment 1. Each of the two mice was then subjected to a preliminary cylinder test for three times.

The experiment results are shown in Table 1 below. The mouse administered with the mixed liquor of embodiment 1 exhibited higher mobility than that administered with the mixed liquor of comparative example 2. In other words, of the two disease-model mice, the one administered with the mixed liquor of embodiment 1 showed a significant relief of PD symptoms (i.e., an increase of mobility) as compared with that administered with the mixed liquor of comparative example 2. This indicates that the disclosed liposome composition can relieve PD symptoms more effectively when containing glutamic acid, DHA, and lecithin as the nutrients than when containing only glutamic acid as the nutrients.

TABLE 1

| | Disease-model | Disease-model |
|---|---|---|
| | mouse | mouse |
| | administered | administered |
| | with mixed liquor | with mixed |
| | of comparative | liquor of |
| | example 2 | embodiment 1 |
| Average number of | 24 times | 33 times |
| times the forelimbs | | |
| of the mouse touched | | |
| the cylinder wall | | |
| within five minutes | | |

[Experiment 3]: Survival Analysis

To test for any lethal side effect of the present invention on mice, experiment 3 was carried out by feeding the mixed liquor of embodiment 1 and the mixed liquor of comparative example 1 separately to disease-model mice and healthy-model mice of the same litter.

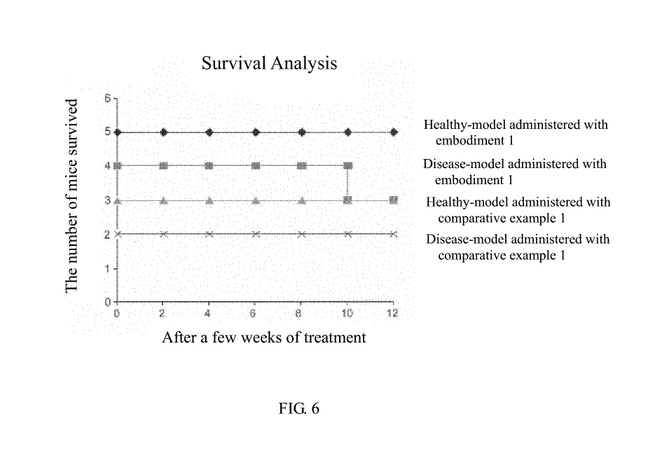

The results of the survival analysis are plotted in FIG. 6. Only one mouse died 10 weeks after persistent treatment with the mixed liquor of embodiment 1 (10 mg/day for two consecutive weeks). All the other mice survived, be they healthy or disease models, administered with the mixed liquor of embodiment 1 or comparative example 1. Neither immediate death associated with the treatment nor clearly abnormal behaviors were found among the surviving mice. The analysis results show that persistent treatment with the mixed liquor of embodiment 1 (10 mg/day for two consecutive weeks) did not cause immediate death of the treated mice.

Therefore, the liposome composition disclosed herein has a phospholipid bilayer and a special targeting substance that can pass through the BBB of a living body and then release nutrients, including glutamic acid, to activate glial cells in the living body, in order for the glial cells to acquire calcium ions in the living body, the goal being to promote synaptogenesis and to modulate neuronal plasticity and synaptic transmission while enhancing the glutamic acid-glutamine cycle.

The present invention is more detailed illustrated by the above preferable example embodiments. While example embodiments have been disclosed herein, it should be understood that other variations may be possible. Such variations are not to be regarded as a departure from the spirit and scope of example embodiments of the present application, and all such modifications as would be obvious to one skilled in the art are intended to be included within the scope of the following claims.

What is claimed is:

1. A liposome composition, comprising:
a liposome enclosed by a phospholipid bilayer, wherein the phospholipid bilayer defines a receiving space therein;
nutrients provided in the receiving space, wherein the nutrients comprising glutamic acid, docosahexaenoic acid (DHA), and lecithin; and
a targeting substance embedded in or bound to the liposome.

2. The liposome composition of claim 1, wherein the liposome is selected from the group consisting of: a liposome, a phytosome, an ethosome, a phosphatidylcholine, a phosphatidylserine, and a phosphatidylinositol.

3. The liposome composition of claim 1, wherein the liposome comprising a plurality of phospholipid bilayers that are enclosed to form a plurality of receiving spaces containing nutrients.

4. The liposome composition of claim 1, wherein the nutrients further comprises a vitamin, an antioxidant, or a mixture or a complex thereof.

5. The liposome composition of claim 1, wherein the targeting substance is a vitamin E derivative, or a lipid derivative group, bound with a polyethylene glycol (PEG), or a derivative thereof, and glutathione (GSH), or a derivative thereof.

6. The liposome composition of claim 5, wherein the liposome is a targeting glutathione transporter.

7. The liposome composition of claim 5, wherein the liposome is a glutathione transporter targeted at the BBB.

8. The liposome composition of claim 5, wherein the PEG or PEG derivative comprises a carboxylic acid, maleimide, amide, or biotin.

9. The liposome composition of claim 1, wherein the targeting substance is polysorbate nanoparticles.

10. The liposome composition of claim 1, wherein the targeting substance is an amino acid sequence.
